(19) United States
(12) Patent Application Publication
(10) Pub. No.: US 2011/0152346 A1
(43) Pub. Date: Jun. 23, 2011

(54) Use of Oligonucleotides with Modified Bases in Hybridization of Nucleic Acids

(75) Inventors: Mati Karleson, Tartu (EE); Erkki Truve, Tallinn (EE); Allan Olspert, Harjumaa (EE); Cecilia Sarmiento, Tallinn (EE); Mart Saarma, Helsinki (FI)

(73) Assignee: Baltic Technology Development Ltd., Tallinn (EE)

(21) Appl. No.: 12/741,527

(22) PCT Filed: Nov. 5, 2008
(86) PCT No.: PCT/FI08/50635

§ 371 Date: Feb. 14, 2011

Related U.S. Application Data
(60) Provisional application No. 60/985,552, filed on Nov. 5, 2007.

(51) Int. Cl. A61K 31/7088; C12N 5/07; C12P 19/34; C07H 21/00; C12Q 1/68; G01N 33/53; A61P 37/02; A61P 31/12; A61P 35/00

(52) U.S. Cl. 514/44 A; 435/375; 435/91.2; 536/23.1; 435/6.12; 436/94

(57) ABSTRACT

The invention is concerned with the use of oligonucleotide analogs that contain specifically modified DNA bases to be used in hybridization of nucleic acids, polymerase chain reaction (PCR) and siRNA-mediated gene silencing (RNAi).

### CROSS-REFERENCE TO RELATED APPLICATION

This application claims the benefit of prior U.S. provisional application No. 60/985,552 filed Nov. 5, 2007, hereby incorporated by reference in its entirety.

### FIELD OF THE INVENTION

The invention is concerned with the use of modified oligonucleotides that contain specifically modified DNA bases to be used in hybridization of nucleic acids, amplifying nucleic acids (e.g., with polymerase chain reaction (PCR)) and siRNA-mediated gene silencing (RNAi).

### BACKGROUND OF THE INVENTION

The use of oligonucleotides and modified oligonucleotides is of great significance in modern therapy and has been well documented (Uhlmann, et al. Antisense oligonucleotides: A new therapeutic principle. Chemical Reviews 1990, 90: 543-584; Crooke, et al. “Antisense Research and Applications”, CRC Press (1993); Mesmaekar, et al. “Antisense oligonucleotides,” Acc. Chem. Res. 1995, 28: 366-374; Stein. “The experimental use of antisense oligonucleotides: a guide for the perplexed.” J. Clin. Invest. 2001, 108, 641-644). The specific binding of antisense polynucleotides to the DNA or RNA targets can inactivate the replication, transcription, or translation of nucleic acids, thereby providing a mechanism for controlling diseases such as cancer and viral infection. The binding of an antisense oligonucleotide to a target can thus be used to alter gene expression, in a variety of circumstances, e.g., to interfere with viral life cycles, or the growth of cancerous cells.

Arrays of binding oligonucleotides have become an increasingly important tool in the biotechnology industry and related fields. These arrays, deposited onto a solid support surface, are applied in many fields, including drug screening, nucleic acid sequencing, mutation analysis, etc.

For instance, nucleic acid hybridization has become an increasingly important means of identifying, measuring and detecting the presence of particular nucleic acids in a given sample. Therefore, medical diagnostics, forensics, environmental and food testing, have all benefited from the use of nucleic acid hybridization as a rapid, simple and accurate way of testing for the presence or absence of given biological contaminants or microorganisms in a sample. Mechanistically, nucleic acid hybridization exploits the ability of single-stranded nucleic acids to form stable hybrids with corresponding regions of nucleic acid strands having complementary nucleotide sequences. Such hybrids usually consist of double-stranded duplexes, although triple-stranded structures are also known. In a nucleic acid duplex, each base pair contributes to stability. Hence, the shorter the duplex, the greater the relative contribution of each individual base pair to the stability of the duplex. As a result, the difference in stability between a perfect match and a mismatch will be greater for shorter oligonucleotides. However, as short oligonucleotides hybridize weakly, it could be enhanced by using more strongly binding nucleotides.

A number of methods have been developed for exponential amplification of nucleic acids. These include the polymerase chain reaction (PCR), ligase chain reaction (LCR), self-sustained sequence replication (3SR), nucleic acid sequence based amplification (NASBA), strand displacement amplification (SDA), and amplification with Q-Beta replicase. The success of PCR depends on the efficiency the primer binding to the DNA single strain. Again, the stronger the binding, the more DNA will be amplified in a given cycle.

RNA-induced gene silencing in mammalian cells is presently believed to implicate a minimum of three different levels of control: (i) transcription inactivation (siRNA-guided DNA and histone methylation); (ii) small interfering RNA (siRNA)-induced mRNA degradation; and (iii) mRNA-induced transcriptional attenuation. The RNA interference (RNAi) generated by siRNA can be long lasting and effective over multiple cell divisions. Consequently, the ability to assess gene function via siRNA mediated methods, as well as to develop therapies for over-expressed genes, represents an exciting and valuable tool that will accelerate gene function analysis, drug target validation, and genome-wide investigations.

In all the above areas there have been attempts to increase the efficiency by employing modified nucleotides. Thus, a number of oligonucleotide derivatives have been constructed having modifications at the nitrogenous base, including replacement of the amino group at the 6 position of adenosine by hydrogen to yield purine; substitution of the 6-keto oxygen of guanosine with hydrogen to yield 2-amino purine, or with sulphur to yield 6-thioguanosine, and replacement of the 4-keto oxygen of thymidine with either sulphur or hydrogen to yield, respectively, 4-thiothymidine or 4-hydrothymidine. All these nucleotide analogues can be used as reactants for the synthesis of oligonucleotides. Similarly, a number of nucleotide derivatives have been reported having modifications of the ribofuranosyl or deoxyribofuranosyl moiety. Most oligonucleotides comprising such modified bases have been formulated with increased cellular uptake, nuclease resistance, and/or increased substrate binding in mind.

### SUMMARY OF THE INVENTION

The present invention relates to oligonucleotides that comprise modified nucleobases, which increase their binding ability to complementary nucleic acids and can increase the hybridization of them with nucleic acid complementary strands. Depending on the nature of the number of modified nucleobases in the oligonucleotide portion of the disclosed compounds, the binding ability of the compound to a complementary target nucleic acid can be increased manifold, compared to a typical complementary oligonucleotide.

The use of such modified oligonucleotide analogs in hybridization of nucleic acids, polymerase chain reaction (PCR) and siRNA-mediated gene silencing (RNAi) is the subject of the present invention. One aspect of the invention is an oligonucleotide analog used as a labeled probe for nucleic acid hybridization. This hybridization includes, among others, hybridization of the probe to DNA (e.g. Southern hybridization), to RNA (e.g. Northern hybridization), hybridization of the probe to any nucleic acid sequence attached to a chip, etc. Another aspect of the invention is one oligonucleotide analog or a pair of or pairs of oligonucleotide analogs used as PCR primers during the PCR reaction. These PCR reactions involve, as an example, conventional PCR, real-time PCR, reverse-transcription PCR, etc., in general all different reactions where repetitive catalytic synthesis of phoshodiester bonds takes place, interrupted by the denaturation of previously synthesized double-stranded nucleic acid strands with high temperature. Another aspect of the invention is an oligoribonucleotide analog used together with an unmodified oligoribonucleotide or, alternatively with another modified oligoribonucleotide, to anneal a short-interfering RNA (siRNA) and use the annealed siRNA to silence the nucleic acid sequence complementary to the corresponding siRNA. The mentioned siRNA can be introduced into the cell using transfection, microinjection, bombardment, viral vectors or other techniques. The mentioned silencing means for example sequence-specific degradation of the target nucleic acid or sequence-specific translation inhibition of the target nucleic acid.

In view of the exceptional binding power of the modified oligonucleotides of the present invention, one aspect of the invention is a method of inhibiting expression of a target nucleic acid comprising contacting a target nucleic acid of known sequence with a modified oligonucleotide having a sequence of nucleobases that is at least partly complementary to a strand of said target nucleic acid, under conditions that permit hybridizing of the modified oligonucleotide to a strand of the target nucleic acid, wherein the hybridized modified oligonucleotide inhibits expression of the target nucleic acid, wherein the modified oligonucleotide comprises from 5 to 150 nucleobases, and wherein at least one of the nucleobases of the modified oligonucleotide is a modified nucleobase selected from the group consisting of: 5-mercaptocytosine, 5-mercaptouracil, 8-mercaptoguanine, 8-mercaptoadenine, 5-hydroxycytosine, 5-hydroxyuracil, 8-hydroxyadenine and 8-hydroxyguanine.

In one aspect, the expression of a target nucleic acid is inhibited by at least 20%. In some aspects the target nucleic acid is RNA, and in further aspects the modified oligonucleotide is single-stranded in nature. In some aspects, the modified oligonucleotide is used with another oligonucleotide and is double-stranded wherein at least one strand of the two strands is a modified oligonucleotide that comprises the at least one modified nucleobase.

Yet another aspect of the present invention relates to the target nucleic acid being in a cell, and the contacting of the modified oligonucleotide to the target nucleic acid comprises introducing the modified oligonucleotide into the cell. In some of these aspects, the contacting is selected from the group consisting of transforming and transfecting the cell with the modified oligonucleotide.

In still other aspects, the target nucleic acid is in a cell of an organism, and the contacting comprises administering to the organism a composition that comprises the modified oligonucleotide and a pharmaceutically acceptable carrier. In some embodiments, the composition further comprises a delivery vehicle, such as a liposome. The organism in various aspects is a mammal, and in other aspects is a human.

In addition to the foregoing, the invention includes, as an additional aspect a method of detecting a target nucleic acid with a modified oligonucleotide comprising contacting a target nucleic acid with a modified oligonucleotide under conditions that permit hybridizing of the modified oligonucleotide to a strand of said target nucleic acid (which may have 1, 2, or more strands, usually 1 or 2), wherein the modified oligonucleotide comprises a sequence of nucleobases that is at least partly complementary to a sequence of the strand of the target nucleic acid, and wherein at least one of the nucleobases of the modified oligonucleotide is a modified nucleobase selected from the group consisting of: 5-mercaptocytosine, 5-mercaptouracil, 8-mercaptoguanine, 8-mercaptoadenine, 5-hydroxycytosine, 5-hydroxyuracil, 8-hydroxyadenine and 8-hydroxyguanine; and detecting the target nucleic acid by detecting the modified oligonucleotide hybridized to the strand of the target nucleic acid.

In some aspects, the target nucleic acid is immobilized to a solid support. In additional aspects the immobilized target nucleic acid is DNA or RNA. In some of the foregoing aspects, the detecting is quantitative in nature. For example, a measurement of the hybridization provides an absolute or relative measure of the amount of the target nucleic acid.

A further aspect of the present invention is a method of polymerase chain reaction (PCR) comprising contacting a template nucleic acid with a modified oligonucleotide comprising a sequence sufficiently complementary to a portion of the template nucleic acid to allow hybridization of the modified oligonucleotide with the template nucleic acid under PCR annealing conditions, wherein the hybridized modified oligonucleotide serves as a PCR primer under PCR amplification conditions to generate a first strand PCR product, and wherein the modified oligonucleotide comprises from 5 to 150 nucleobases, wherein at least one of the nucleobases is a modified nucleobase selected from the group consisting of: 5-mercaptocytosine, 5-mercaptouracil, 8-mercaptoguanine, 8-mercaptoadenine, 5-hydroxycytosine, 5-hydroxyuracil, 8-hydroxyadenine and 8-hydroxyguanine.

In some aspects, the PCR comprises making a reaction mixture that contains a thermostable DNA polymerase, the template nucleic acid, the modified oligonucleotide, and nucleotides (e.g., dATP, dTTP, dCTP, dGTP). Reagents used in PCR reactions, including MgCl₂, buffers, and the like are well known.

In still other aspects, the PCR reaction mixture further comprises a second oligonucleotide that comprises a nucleotide sequence complementary to at one of a part of a strand of the target nucleic acid or a part of the first strand PCR product. In various aspects, the second oligonucleotide is a modified oligonucleotide, wherein the modified oligonucleotide comprises from 5 to 150 nucleobases, wherein at least one of the nucleobases is a modified nucleobase selected from the group consisting of: 5-mercaptocytosine, 5-mercaptouracil, 8-mercaptoguanine, 8-mercaptoadenine, 5-hydroxycytosine, 5-hydroxyuracil, 8-hydroxyadenine and 8-hydroxyguanine. In some aspects the template nucleic acid is DNA while in other aspects the template nucleic acid is RNA.

The present invention also provides methods wherein the amplified product is quantified in real time.

In still further aspects, the polymerase chain reaction comprises repeated steps of denaturing the template nucleic acid, annealing the modified oligonucleotide and template nucleic acid under annealing conditions, and synthesizing a polymerase chain reaction product by extending the annealed modified oligonucleotide.

In some embodiments of the invention, the modified oligonucleotide comprises a detectable label.

The methods of the invention provide that in some aspects, the hybridizing conditions comprise a pH of between 4 and 10. In other aspects, the hybridizing conditions comprise a pH of between 4 and 6.

In some variations of the invention, the modified oligonucleotides as provided by the present invention are contemplated to have a length of from 10 to 100 nucleobases. In various aspects, the modified oligonucleotide has a length of from 10 to 50 nucleobases. In still further aspects, the modified oligonucleotide has a length of from 20 to 30 nucleobases.

In some aspects, from 0.5% to 40% of the nucleobases of the modified oligonucleotide comprise mercapto- or hydroxynucleobases.

In addition to the foregoing, the invention includes, as an additional aspect, all embodiments of the invention narrower in scope in any way than the variations specifically mentioned above. For example, although aspects of the invention may have been described by reference to a genus or a range of values for brevity, it should be understood that each member of the genus and each value or sub-range within the range is intended as an aspect of the invention. Likewise, various aspects and features of the invention can be combined, creating additional aspects which are intended to be within the scope of the invention.

Aspects of the invention described in the singular (including the use of articles “a” or “an” should be understood to include embodiments involving one or more than one, unless context clearly requires a narrower interpretation. The term “comprising” is intended to be permissive of additional elements or features.

Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1 depicts the polynomial fitting of the relative binding efficiency of the modified oligonucleotides at a concentration of 1 pmol.

FIG. 2 depicts the polynomial fitting of the relative binding efficiency of the modified oligonucleotides at a concentration of 5 pmol.

FIG. 3 depicts the polynomial fitting of the relative binding efficiency of the modified oligonucleotides to 1 ng DNA.

FIG. 4 depicts the polynomial fitting of the relative binding efficiency of the modified oligonucleotides to 5 ng DNA.

FIG. 5 depicts the efficiency of modified oligonucleotides in hybridization.

FIG. 6 depicts the polynomial fitting of the relative binding efficiency of the modified oligonucleotides to 1.25 ng of complementary mRNA.

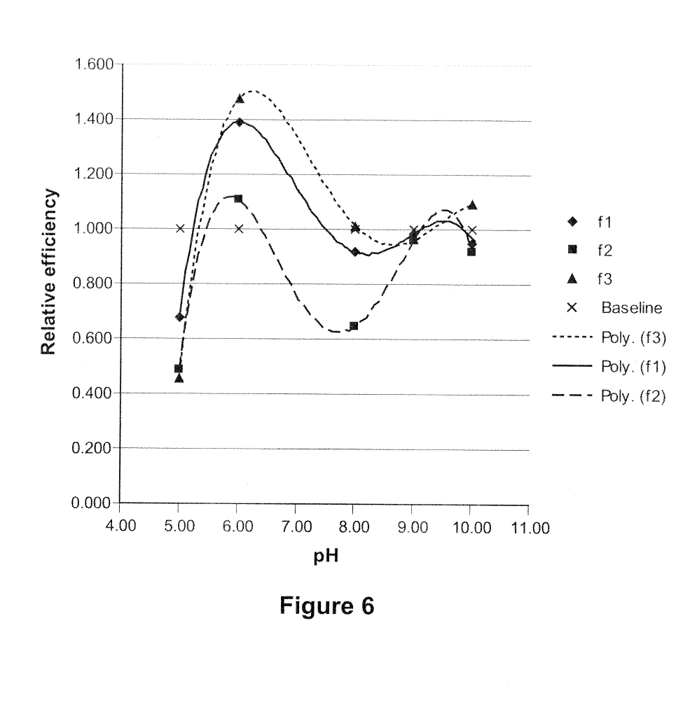

FIG. 7 depicts the polynomial fitting of the relative binding efficiency of the modified oligonucleotides to 2.5 ng of complementary mRNA.

FIG. 8 depicts the polynomial fitting of the relative binding efficiency of oligonucleotide f1 at different pH values for different target concentrations.

FIG. 9 depicts the polynomial fitting of the relative binding efficiency of oligonucleotide f2 at different pH values for different target concentrations.

FIG. 10 depicts the usage of modified oligonucleotides in PCR. The applicability and efficacy of the oligonucleotide with modified bases relative to increasing annealing temperature in PCR is shown.

FIG. 11 depicts the effect of modified siRNAs on eGFP transgene expression.

### DETAILED DESCRIPTION OF THE INVENTION

The present invention provides novel compounds comprising an oligonucleotide having properties for use in antisense and other methods employing oligonucleotides. The compounds of the invention include antisense and other oligonucleotides having one or more modified nucleobases having high binding efficiency to natural nucleobases. The compounds comprising the modified oligonucleotides are useful in the hybridization of nucleic acids, PCR and siRNA-mediated gene silencing (RNAi).

### Oligonucleotides

In the context of this invention, the term “oligonucleotide” refers to an oligomer or polymer of deoxyribonucleic acid (DNA) or ribonucleic acid (RNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function in a similar manner as naturally occurring oligonucleotides when, e.g., hybridizing to target nucleic acids or interacting with complementary oligonucleotides. Such modified or substituted oligonucleotides are often preferred over native forms because of desirable properties such as, for example, enhanced cellular uptake, enhanced affinity for a target nucleic acid and increased stability in the presence of nucleases.

The enhanced efficiency of binding of compounds of the present invention to biological counterparts (e.g., RNA and/or DNA) is attained via incorporation of modified nucleobases or other analogs having zwitterionic or ionic tautomers. Compounds of the present invention have at least one nucleobase having modified nucleobases or other analogs having zwitterionic or ionic tautomers. In preferred embodiments, the modified nucleobase is a hydroxynucleobase selected from 5-hydroxycytosine and 8-hydroxyguanine or a mercaptonucleobase selected from 5-mercaptocytosine, 5-mercaptouracil, 8-mercaptoguanine, and 8-mercaptoadenine. As demonstrated in U.S. patent publication 20070259830 and International Patent Publication WO 2007/125173, both incorporated by reference, a more stable hydrogen bonding of a hydroxynucleobase or mercaptonucleobase with a nucleobase of a target nucleic acid can occur, and, therefore, those can be considered as more effectively binding to a complementary nucleic acid strand.

The acidic tautomeric group in the modified nucleobases can be any other acidic group such as the —SH, —COOH, —SO3H, etc.

In one embodiment, an oligonucleotide comprises one or more tautomeric forms of the 5-hydroxyuracil anion. In another embodiment, the compounds of the present invention include the hydroxybase 5-hydroxycytosine. In another embodiment, the hydroxybase is a tautomeric form of the 8-hydroxyadenine and its anion. Another embodiment of the invention provides compounds of the present invention modified by tautomeric forms of the 8-hydroxyguanine and its anion. Tautomeric forms of these nucleobases are described in further detail in WO 2007/125173, incorporated herein by reference.

Additional modified nucleobases contemplated herein include mercapto-modified nucleobases. Synthesis of mercapto-modified pyrimidines and purines is known in the art (See for example, “Chemistry of Heterocyclic Compounds: The Pyrimidines,” Supplement 1, Volume 16, Editor D. J. Brown, John Wiley & Sons, Inc., 1970, pp. 202-229; and Khalyullin et al., “Condensed purines”, Pharmaceutical Chemistry Journal, 1992, 26: 270-284). Mercaptonucleobases contemplated include 5-mercaptocytosine, 5-mercaptouracil, 8-mercaptoguanine and 8-mercaptoadenine.

As used herein, each of the hydroxynucleobases is considered complementary to a nucleobase when it stably hydrogen bonds to the opposite nucelobase. Therefore, in some cases, 5-hydroxyuracil is complementary to adenine, 5-hydroxycytosine is complementary to guanine, 8-hydryoxyadenine is complementary to uracil and/or thymine, and 8-hydroxyguanine is complementary to cytosine. Other stable hydrogen bonding of a hydroxynucleobase with a nucleobase of a target nucleic acid can occur, and, therefore, a hydroxynucleobase is considered complementary to the nucleobases of the target nucleic acid to which stable hydrogen bonding occurs.

The number of hydroxynucleobases and/or mercaptonucleobases in a given compound of the present invention is at least 1% up to 100% of the total number of nucleobases of the oligonucleotide portion of the compound. In cases where more than one hydroxynucleobase or mercaptonucleobase is present in the compounds of the present invention, the hydroxynucleobases of mercaptonucleobases may be the same or different (in any combination of different bases and/or types of modifications). It is contemplated that 10% to 90%, 20% to 80%, 30% to 70%, 40% to 60% or 50% of the nucleobases in an oligonucleotide described herein are modified nucleobases. It is further contemplated that 10, 20, 30, 40, 50, 60, 70, 80, 90, 91, 92, 93, 94,95, 96, 97, 98, or 99% of the nucleobases are modified nucleobases.

The compounds in accordance with this invention preferably comprise from about 5 to about 150 nucleobases (i.e. from about 5 to about 150 linked nucleosides). One of ordinary skill in the art will appreciate that the invention embodies compounds of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, and 150 nucleobases in length.

In one preferred embodiment, the compounds of the invention are 10 to 100 nucleobases in length. One having ordinary skill in the art will appreciate that this embodies compounds of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleobases in length.

In another preferred embodiment, the compounds of the invention are 10 to 50 nucleobases in length. One having ordinary skill in the art will appreciate that this embodies compounds of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length.

In another preferred embodiment, the compounds of the invention are 20 to 30 nucleobases in length. One having ordinary skill in the art will appreciate that this embodies compounds of 20, 21, 22, 23, 24, 25, 26, 27, 28, 29, or 30 nucleobases in length.

Particularly preferred compounds are oligonucleotides from about 10 to about 50 nucleobases, even more preferably those comprising from about 20 to about 30 nucleobases, the compounds used in sample tests as antiviral agents were comprised from 21 or from 23 nucleobases.

As stated above, the oligonucleotide may contain 100% modified nucleobases. As such, depending on the length of the oligonucleotide, the oligonucleotide may contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 modified nucleobases, wherein the modified base is either a mercaptonucleobase or an hydroxynucleobase.

The compound of the present invention optionally further comprises a chelating moiety. Chelating moieties function as metal ligands. They can stably chelate a metal ion. Certain metal-ligand complexes have been shown to be effective in cleaving phosphodiester bonds. In incorporating a chelating moiety into an oligonucleotide capable of antisense activity, the efficacy of the oligonucleotide in inhibiting a target nucleic acid increases due to its ability to degrade or cleave one or more phosphodiester bonds of the target nucleic acid. Therefore, the compounds of the present invention further comprise chelating moieties capable of chelating a metal ion. The metal ion is selected from the group consisting of lanthanum, cerium, praseodymium, neodymium, promethium, samarium, europium, gadolinium, terbium, dysprosium, holmium, erbium, thulium, ytterbium, and lutetium. In one aspect, preferred ions are ions of europium or lanthanum. Ions of the metals can be any stable ion, such as +1, +2, +3, +4, or +5. Preferred ions are La(III), Eu(III), Ho(III), and Ce(IV).

Contemplated chelating moieties include those represented by formulas as described below.

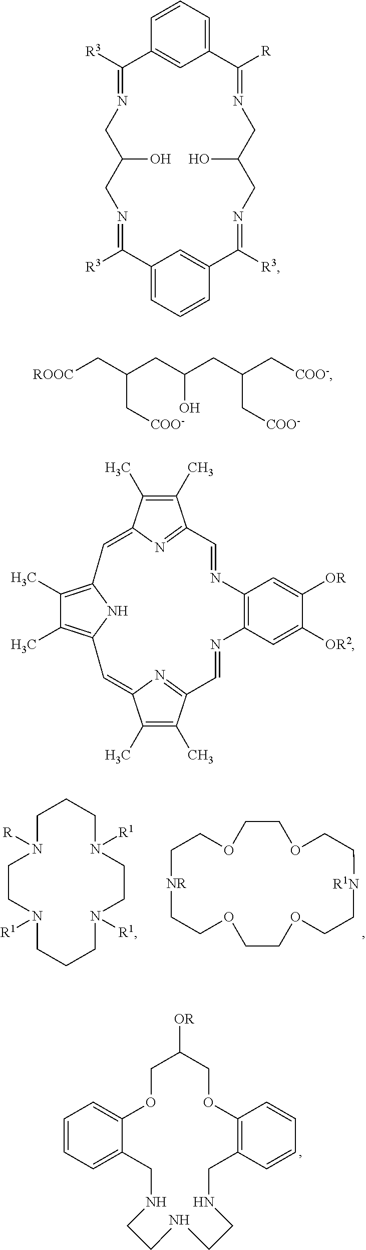

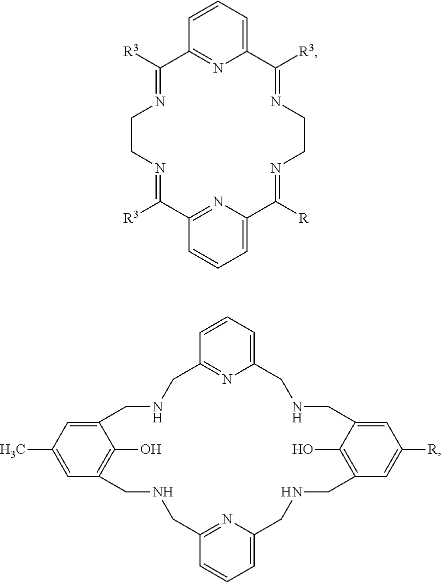

where R is the rest of the oligonucleotide;

R1 is selected from hydrogen, C1-8 alkane, C2-8 alkene, C2-8 alkyne, acylC1-8alkane, cycloalkyl, heterocycloalkyl, aryl, heteroaryl, C1-8alkylaryl, and C1-8alkylheteroaryl

R2 is independently selected from C1-8 alkyl, C2-8 alkene, C2-8 alkyne, aryl, heteroaryl, C1-8alkylaryl, C1-8alkylheteroaryl, and acylC1-8alkane, and

R3 is independently selected from the group consisting of hydrogen, C1-8 alkane, C2-8 alkene, C2-8 alkyne, acylC1-8alkane, cycloalkyl, heterocycloalkyl, aryl, heteroaryl, C1-8alkylaryl, and C1-8alkylheteroaryl.

The term “alkyl” includes straight chained and branched hydrocarbon groups containing the indicated number of carbon atoms, typically methyl, ethyl, and straight chain and branched propyl and butyl groups. The hydrocarbon group can contain up to 16 carbon atoms. The term “alkyl” includes “bridged alkyl,” e.g., a C6-C16 bicyclic or polycyclic hydrocarbon group, for example, norbornyl, adamantyl, bicyclo[2.2.2]octyl, bicyclo[2.2.1]heptyl, bicyclo[3.2.1]octyl, and decahydronaphthyl.” The term “alkyl” also encompasses alkyl groups which are optionally substituted with, e.g., one or more halogen atoms, one or more hydroxyl groups, or one or more thiol groups. The term “cycloalkyl” is defined as a cyclic C3-C8 hydrocarbon group, e.g., cyclopropyl, cyclobutyl, cyclohexyl, and cyclopentyl. “Heterocycloalkyl” is defined similar to cycloalkyl, except at least one heteroatom is present in the cyclic structure. Suitable heteroatoms include N, S, and O.

The terms “alkenyl” and “alkynyl” are defined identically as “alkyl,” except for containing a carbon-carbon double bond or carbon-carbon triple bond, respectively. “Cycloalkenyl” is defined similarly to cycloalkyl, except a carbon-carbon double bond is present in the ring.

The term “alkylene” refers to an alkyl group having a substituent. For example, the term “C1-3alkylenearyl” refers to an alkyl group containing one to three carbon atoms, and substituted with an aryl group.

The term “halo” or “halogen” is defined herein to include fluorine, bromine, chlorine, and iodine.

The term “aryl,” alone or in combination, is defined herein as a monocyclic or polycyclic aromatic group, preferably a monocyclic or bicyclic aromatic group, e.g., phenyl or naphthyl. Unless otherwise indicated, an “aryl” group can be unsubstituted or substituted, for example, with one or more, and in particular one to three, halo, alkyl, hydroxy, C(═O)OR, hydroxyalkyl, alkoxy, alkoxyalkyl, haloalkyl, haloalkoxy, cyano, nitro, amino, alkylamino, acylamino, alkylthio, alkylsulfinyl, and alkylsulfonyl. Exemplary aryl groups include phenyl, naphthyl, tetrahydronaphthyl, 2-chlorophenyl, 3-chlorophenyl, 4-chlorophenyl, 2-methylphenyl, 4-methoxyphenyl, 3-trifluoromethylphenyl, 4-nitrophenyl, and the like. The terms “arylC1-3alkyl” and “heteroarylC1-3alkyl” are defined as an aryl or heteroaryl group having a C1-3alkyl substituent.

The term “heteroaryl” is defined herein as a monocyclic or bicyclic ring system containing one or two aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, alkylamino, acylamino, alkylthio, alkylsulfinyl, and alkylsulfonyl. Examples of heteroaryl groups include thienyl, furyl, pyridyl, oxazolyl, quinolyl, isoquinolyl, indolyl, triazolyl, isothiazolyl, isoxazolyl, imidizolyl, benzothiazolyl, pyrazinyl, pyrimidinyl, thiazolyl, and thiadiazolyl.

The term “Het” is defined as monocyclic, bicyclic, and tricyclic groups containing one or more heteroatoms selected from the group consisting of oxygen, nitrogen, and sulfur. A “Het” group also can contain an oxo group (═O) attached to the ring. Nonlimiting examples of Het groups include 1,3-dioxolanyl, 2-pyrazolinyl, pyrazolidinyl, pyrrolidinyl, piperazinyl, a pyrrolinyl, 2H-pyranyl, 4H-pyranyl, morpholinyl, thiopholinyl, piperidinyl, 1,4-dithianyl, and 1,4-dioxane.

The term “hydroxyl” is defined as —OH.

The term “alkoxy” is defined as —OR, wherein R is alkyl.

The term “alkoxyalkyl” is defined as an alkyl group wherein a hydrogen has been replaced by an alkoxy group. The term “(alkylthio)alkyl” is defined similarly as alkoxyalkyl, except a sulfur atom, rather than an oxygen atom, is present.

The term “hydroxyalkyl” is defined as a hydroxy group appended to an alkyl group.

The term “amino” is defined as —NH2, and the term “alkylamino” is defined as —NR2, wherein at least one R is alkyl and the second R is alkyl or hydrogen.

The term “acylamino” is defined as RC(═O)N—, wherein R is alkyl or aryl.

The term “alkylthio” is defined as —SR, wherein R is alkyl.

The term “alkylsulfinyl” is defined as RSO2-, wherein R is alkyl.

The term “alkylsulfonyl” is defined as RSO3-, wherein R is alkyl.

The term “nitro” is defined as —NO2.

The term “trifluoromethyl” is defined as —CF3.

The term “trifluoromethoxy” is defined as —OCF3.

The term “cyano” is defined as —CN.

The calculated nuclease efficiency of a compound of the present invention comprising a chelating moiety complexed to a metal ion increases, depending on the nature of the number of modified nucleobases, up to 10³-10⁹ times in comparison to naturally-occurring nucleases, allowing a corresponding lowering of the effective concentration, and keeping at the same time high specificity of the compound.

Other modifications of compounds of this invention are also contemplated. While oligonucleotides are a preferred form of the compound of the invention, the present invention comprehends other families of compounds, including, but not limited to oligonucleotide analogs and mimetics.

Additional antisense compounds contemplated for use in the compositions and methods of the invention, include but are not limited to, oligonucleotides containing modified backbones (e.g., with or without a phosphorous atom) or non-natural internucleoside linkages, oligonucleosides, modified oligonucleotide backbones that do not include a phosphorus atom which have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages (e.g., morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts), oligonucleotides having inverted polarity, oligonucleotide mimetics, optionally in which both the sugar and the internucleoside linkage (i.e. the backbone) of the nucleotide units are replaced with novel groups, peptide nucleic acid (PNA), oligonucleotides having one or more substituted sugar moieties, including but not limited to, one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl, Locked Nucleic Acids (LNAs) in which the 2′-hydroxyl group is linked to the 3′ or 4′ carbon atom of the sugar ring, thereby forming a bicyclic sugar moiety, oligonucleotides with synthetic and natural nucleobases, including but not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone, oligonucleotides chemically linked to primary or secondary hydroxyl groups, including but not limited to, chelating moieties, intercalators, reporter molecules, polyamines, polyamides, polyethylene glycols, polyethers, groups that enhance the pharmacodynamic properties of oligomers, and groups that enhance the pharmacokinetic properties of oligomers. The modifications set out above are further described in WO 2007/125173, incorporated herein by reference.

Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid.

### Further Modifications of Compounds of the Invention

Other modifications of compounds of this invention are also contemplated. While oligonucleotides are a preferred form of the compounds of this invention, the present invention comprehends other families of compounds as well, including but not limited to oligonucleotide analogs and mimetics such as those described herein.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

### Modified Internucleoside Linkages (Backbones)

Specific examples of contemplated antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Contemplated modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Contemplated oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is herein incorporated by reference.

Contemplated modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

### Modified Sugar and Internucleoside Linkages-Mimetics

In other contemplated oligonucleotide mimetics, both the sugar and the internucleoside linkage (i.e. the backbone), of the nucleotide units are replaced with novel groups. The nucleobase units are maintained for hybridization with an appropriate target nucleic acid. One such compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254:1497-1500.

Certain embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— (wherein the native phosphodiester backbone is represented as —O—P—O—CH₂—) of the above referenced U.S. Pat. No. 5,489,677, and the amide backbones of the above referenced U.S. Pat. No. 5,602,240. Also contemplated are oligonucleotides having morpholino backbone structures of the above-referenced U.S. Pat. No. 5,034,506.

### Modified Sugars

Modified oligonucleotides may also contain one or more substituted sugar moieties. Contemplated oligonucleotides comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are ORCH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A contemplated modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further contemplated modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂, also described in examples hereinbelow.

Other contemplated modifications include 2′-methoxy (2′-O—CH₃), 2′-aminopropoxy (2′-OCH₂CH₂CH₂NH₂), 2′-allyl (2′-CH₂—CH═CH₂), 2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is herein incorporated by reference in its entirety.

A further preferred modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is preferably a methylene (—CH₂-)n group bridging the 2′ oxygen atom and the 4′ carbon atom wherein n is 1 or 2. LNAs and preparation thereof are described in WO 98/39352 and WO 99/14226.

### Natural and Modified Nucleobases

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, ed. John Wiley & Sons, 1990, those disclosed by Englisch et al., Angewandte Chemie, International Edition, 1991, 30:613, and those disclosed by Sanghvi, Chapter 15, Antisense Research and Applications, pages 289-302, Crooke and Lebleu, ed., CRC Press, 1993.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, each of which is herein incorporated by reference, and U.S. Pat. No. 5,750,692, also herein incorporated by reference.

Modified nucleobases have also been contemplated for use as antiviral agents in co-owned and co-pending application Ser. No. ______ (Attorney Docket Number 28113/43434B), as well as U.S. provisional application Nos. 60/985,548 filed on Nov. 5, 2007 and 61/057,685 filed on May 30, 2008, which are hereby incorporated by reference in their entirety.

### Antisense Inhibition

The hybridization of a compound of this invention with a target nucleic acid is generally referred to as “antisense.” Such hybridization can lead to inhibition of translation of the target nucleic acid and is termed “antisense inhibition” herein. Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be inhibited include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. In the context of the present invention, “modulation” and “modulation of expression” mean either an increase (stimulation) or a decrease (inhibition) in the amount or levels of a nucleic acid molecule encoding the gene, e.g., DNA or RNA Inhibition is often the preferred form of modulation of expression and mRNA is often a preferred target nucleic acid.

In the context of this invention, “hybridization” means the pairing of complementary strands of oligomeric compounds and is used interchangeably with the term “annealing.” In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

In one aspect of the invention, the expression of a target nucleic acid is inhibited by 20%. In other aspects, the expression of a target nucleic acid is inhibited by at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or at least 99% or more.

In the case of in vitro conditions, the pH at which the hybridization takes place is important. As disclosed herein below, the efficiency with which the modified oligonucleotides of the invention bind to their targets is affected by pH. Highly efficient binding of modified oligonucleotide to target nucleic acid occurs at a pH range of approximately 4 to 10. In one aspect, the pH at which highly efficient binding occurs is about 4. In various aspects, the pH at which highly efficient binding of modified oligonucleotide to target nucleic acid occurs is about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, about 9, about 9.1, about 9.2, about 9.3, about 9.4, about 9.5, about 9.6, about 9.7, about 9.8, about 9.9, or about 10. All ranges defined by these exemplary pH values are specifically contemplated as alternative ways to define in vitro embodiments of the invention.

In the present invention, the phrase “stringent hybridization conditions” or “stringent conditions” refers to conditions under which a compound of the invention will hybridize to its target sequence, but to a minimal number of other sequences. Stringent conditions are sequence-dependent and will be different in different circumstances and in the context of this invention, “stringent conditions” under which oligomeric compounds hybridize to a target sequence are determined by the nature and composition of the oligomeric compounds and the assays in which they are being investigated. One exemplary set of conditions is as follows: Hybridization at 42° C. in 50% formamide, 5×SSC, 20 mM Na.PO₄, pH 6.8; and washing in 1×SSC at 55° C. for 30 minutes. Formulas for calculating equivalent hybridization conditions and/or selecting other conditions to achieve a desired level of stringency are well known. It is understood in the art that conditions of equivalent stringency can be achieved through variation of temperature and buffer, or salt concentration as described Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

“Complementary,” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

It is understood in the art that the sequence of an antisense compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure or hairpin structure). It is preferred that the oligonucleotide portion of the compounds of the present invention comprise at least 70% sequence complementarity to a target region within the target nucleic acid, more preferably that they comprise at least 85% or 90% sequence complementarity, and may comprise at least 95%, 96%, 97%, 98% or 99% sequence complementarity to the target region within the target nucleic acid sequence to which they are targeted. For example, a compound of the present invention in which 18 of 20 nucleobases of the compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, a compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of a compound with a region of a target nucleic acid can be determined routinely using BLAST programs (basic local alignment search tools) and PowerBLAST programs known in the art (Altschul et al., J. Mol. Biol., 1990, 215:403-410; Zhang et al., Genome Res., 1997, 7:649-656). For compounds of the present invention having hydroxynucleobases and/or synthetic analogs (such as other synthetic nucleobases), complementarity can be assessed by the synthetic analogs specificity for a particular nucleobase of the target nucleic acid.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing.

The first evidence that dsRNA could lead to gene silencing in animals came in 1995 from work in the nematode, Caenorhabditis elegans (Guo et al., Cell, 1995, 81:611-620). Montgomery et al. have shown that the primary interference effects of dsRNA are posttranscriptional (Montgomery et al., Proc. Natl. Acad. Sci. USA, 1998, 95:15502-15507). The posttranscriptional antisense mechanism defined in Caenorhabditis elegans resulting from exposure to double-stranded RNA (dsRNA) has since been designated RNA interference (RNAi). This term has been generalized to mean antisense-mediated gene silencing involving the introduction of dsRNA leading to the sequence-specific reduction of endogenous targeted mRNA levels (Fire et al., Nature, 1998, 391:806-811). Recently, it has been shown that it is, in fact, the single-stranded RNA oligomers of antisense polarity of the dsRNAs which are the potent inducers of RNAi (Tijsterman et al., Science, 2002, 295:694-697).

Additional knowledge exists in the art concerning the use of agents, similar to the oligonucleotides but having a different mechanism of action including small interfering RNAs (siRNAs), their precursors and analogs and ribozymes, designed to specifically bind and cleave the target nucleic acids.

### Use of Labeled Oligonucleotide Analogs for Hybridization

The oligonucleotides and compounds described herein can optionally be labeled. One of ordinary skill in the art is capable of labeling an oligonucleotide of the present invention by any of a number of means. The oligonucleotides may be radioactively labeled with ³²P, ³⁵S, or any other radionuclide known to those of skill in the art. Additionally, the oligonucleotides of the present invention may be fluorescently labeled. Fluorescent labels that may be used include, but are not limited to the following: Fluorescein (FITC), CY-5, CY-5.5, CY-3, CY-2, CY-7, Texas Red, Rhodamine, etc.

These labeled modified oligonucleotides are contemplated for use in assays well known in the art, such as Southern and Northern blotting (Sambrook et al., (Eds.), Molecular Cloning: A Laboratory Manual, Cold Spring Harbor Laboratory Press: Cold Spring Harbor, N.Y. (1989)). Another aspect of the invention is the use of the labeled modified oligonucleotides in hybridization to a nucleic acid immobilized on a solid support, such as a chip. One of ordinary skill in the art will understand that these chips can be utilized in microarray studies. Labelled oligonucleotides with modified nucleobases are themselves an aspect of the invention.

A further aspect of the invention is a modified oligonucleotide of the invention or a pair or pairs of modified oligonucleotides used as primers for PCR. The primers may be used for any method of PCR known to those of skill in the art and include, but are not limited to conventional PCR, real-time PCR, reverse transcription PCR (RT-PCR), etc.

It is well-known by those of ordinary skill in the art that PCR comprises the repeated steps of denaturing the target nucleic acid followed by annealing of an oligonucleotide primer to a strand of the denatured target nucleic acid (as described in Sambrook, et al., (Eds.), Molecular Cloning: A Laboratory Manual, Cold Spring Harbor Laboratory Press: Cold Spring Harbor, N.Y. (1989)). This hybridized complex is then extended (i.e., nucleotides are added in succession according to the sequence of the target nucleic acid strand) by the action of a thermostable DNA polymerase. Thermostable DNA polymerases are known in the art and include, but are not limited to Taq, Pfx, and TaKaRa polymerases.

Parameters for PCR such as the temperature at which to conduct annealing and time of extension are empirical in nature and well within the ability of one of ordinary skill in the art to determine.

### Use of Compounds of the Present Invention

The compounds described herein are used in vitro or in vivo for limiting the gene expression and proliferation of pathogens such as viruses, including viruses with DNA genomes, RNA genomes and viruses using reverse transcription. Also see co-owned and co-pending application Ser. No. ______ (Attorney Docket Number 28113/43434B), as well as U.S. provisional application Nos. 60/985,548 filed on Nov. 5, 2007 and 61/057,685 filed on May 30, 2008, which are hereby incorporated by reference in their entirety. Thus, the compounds may be administered to an organism which is subject to or in a diseased state. When administered to an organism, the compounds may be used to treat infection by a variety of pathogens. As used herein “treat” refers to administration of the oligonucleotides of the invention to a subject in need in a dosage/amount sufficient to produce a desired result on a health condition, pathology, and disease of a subject or for a diagnostic purpose. The desired result may comprise a subjective or objective improvement in the recipient of the dosage. “Treatment” refers to prophylactic treatment or therapeutic treatment or diagnostic treatment. A “subject” of diagnosis or treatment is a human or non-human animal, including a mammal or a primate. A “Therapeutically effective amount” refers to that amount of a composition effective to produce the intended beneficial effect on health.

The compounds may be used to modulate the function of immune system cells such as specific B-cells; specific T-cells, such as helper cells, suppressor cells, cytotoxic T-lymphocytes (C), and natural killer (NK) cells. Modulation of immune function using the compounds of the present invention can be useful in treatment of a variety of diseases such as chronic diseases caused by viral pathogens.

The compounds may be selected which are capable of interfering with transcription and/or expression of proteins by any of the mechanisms involved with the binding of the oligonucleotide of the compound to its target sequence. These mechanisms include, but are not limited to, interference with processing, inhibition of transport across the nuclear membrane, cleavage by endonucleases, formation of replicase complexes or the like.

The compounds described herein may be used in the treatment of infectious diseases. The target nucleic acid sequences include, but are not limited to, those genes of pathogenic viruses such as HIV, CMV, HSV, HCV, etc., as well as genes encoding host factors for these viruses or otherwise involved in disease development and/or progression.

In the treatment of cancer, the target nucleic acid sequences can be DNA or RNA associated with oncogenes or viruses with oncogenic properties, tumor suppressor genes, and related genes. Additionally, the compounds of the present invention may also target genes associated with drug resistance and their gene products.

The targeting process usually also includes determination of at least one target region, segment, or site within the target nucleic acid for the antisense interaction to occur such that the desired effect, e.g., modulation of expression, will result. Within the context of the present invention, the term “region” refers to a portion of the target nucleic acid having at least one identifiable structure, function, or characteristic. Within regions of target nucleic acids are segments. “Segments” refers to smaller or sub-portions of regions within a target nucleic acid. “Sites,” as used in the present invention, refer to positions within a target nucleic acid.

It is further contemplated that the compositions described herein are used in combination to hybridize to two different regions or segments within the same viral genome. For choosing a target, the considerations include: localization of the target in a region of the viral genome which is important for virus multiplication (target must be essential region). If possible a preferred target should be in a region which is conserved among different strains and genotypes of the virus (often this also indicates the functional significance of the sequences). The regions encoding highly conserved domains of proteins are good targets; also the regions containing overlapping functional elements (coding sequences overlapping with cis-active elements) are good targets.

It is further contemplated that the target site should have a nucleotide composition that enables construction of an oligonucleotide inhibitor with desirable nucleotide content and/or composition of modified nucleobases, and preferably the target does not contain strong secondary structural elements. Further, the sequence of the target should not overlap with that of essential host genes, especially host mRNAs. Additionally, the positions of modified nucleobases should not match a host sequence. The cluster of C or G nucleotides (three or more) should be avoided. Experiments have shown that target sites inside coding regions are better than those in non-coding regions and that in the case of RNA viruses, the positive strand is a better target than the negative strand. Due to the unique mechanism of nucleic acid destruction (e.g., by RNAse or DNAse complex) it is not necessary to target the modified oligonucleotide to the translation initiation sequence. This is in contrast to the case of morpholino oligonucleotides, which cannot initiate RNA degradation and are most (or exclusively) effective if targeted to the regions containing an initiation codon for translation. Such restriction does not exist for the currently described modified oligonucleotides.

For targeting two or more sites, each site should satisfy several of the criteria set out above. Sequences of the targets should be different and not complementary to each other to avoid aggregation of the oligonucleotide and the targets could represent different sequences from one and the same functional unit, for example from the same enzyme, or, from different units. In most cases the second option is the preferred to minimize the possibility of generation of resistant mutations. As used herein, the term “functional unit” refers to a polypeptide or polynucleotide sequence having a function in viral replication or gene expression, e.g., different replication factors, transcription factors, etc. Oligonucleotides that bind the same functional units bind different target sequences but in the same polypeptide or polynucleotide functional unit, e.g., within the HIV Tat protein. Oligonucleotides contemplated by the invention that bind different functional units bind to polypeptide or polynucleotide having different functions in viral replication or gene expression, e.g., HIV Tat andRev genes or proteins. One of ordinary skill in the art can readily understand the meaning of a functional unit associated with viral replicaion or gene expression.

The translation initiation codon is typically 5′ AUG (in transcribed mRNA molecules; 5′ ATG in the corresponding DNA molecule), the translation initiation codon is also referred to as the “AUG codon,” the “start codon” or the “AUG start codon”. A minority of genes have a translation initiation codon having the RNA sequence 5′ GUG, 5′ UUG or 5′ CUG, and 5′ AUA, 5′ ACG and 5′ CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA transcribed from a gene encoding Interleukin 18, regardless of the sequence(s) of such codons. It is also known in the art that a translation termination codon (or “stop codon”) of a gene may have one of three sequences, i.e., 5′ UAA, 5′ UAG and 5′ UGA (the corresponding DNA sequences are 5′ TAA, 5′ TAG and 5′ TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the antisense compounds of the present invention.

The open reading frame (ORF) or “coding region,” which is known in the art to refer to the region between the translation initiation codon and the translation termination codon, is also a region which may be targeted effectively. Within the context of the present invention, a preferred region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. Targeting splice sites, i.e., intron-exon junctions or exon-intron junctions may also be particularly useful in situations where aberrant splicing is implicated in disease, or where an overproduction of a particular splice product is implicated in disease. Aberrant fusion junctions due to rearrangements or deletions are also preferred target sites. mRNA transcripts produced via the process of splicing of two (or more) mRNAs from different gene sources are known as “fusion transcripts.” It is also known that introns can be effectively targeted using antisense compounds targeted to, for example, DNA or pre-mRNA.

Alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and exonic sequence.

Upon excision of one or more exon or intron regions, or portions thereof during splicing, pre-mRNA variants produce smaller “mRNA variants.” Consequently, mRNA variants are processed pre-mRNA variants and each unique pre-mRNA variant must always produce a unique mRNA variant as a result of splicing. These mRNA variants are also known as “alternative splice variants.” If no splicing of the pre-mRNA variant occurs then the pre-mRNA variant is identical to the mRNA variant.

Variants can be produced through the use of alternative signals to start or stop transcription and pre-mRNAs and mRNAs can possess more than one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Within the context of the invention, the types of variants described herein are also preferred target nucleic acids.

Additional aspects and details of the disclosure will be apparent from the following examples, which are intended to be illustrative rather than limiting.

### EXAMPLES

### Example 1

### Binding of Modified/Unmodified Antisense Oligonucleotides to the Complementary Membrane-Bound Unmodified Sense Oligonucleotides

Each of the following patent applications is incorporated by reference in its entirety: U.S. Ser. No. 60/797,448, filed May 3, 2006, and Ser. No. 11/742,384, filed Apr. 30, 2007 (published as US Patent Application Publication No. 2007/0259830).

In order to compare the relative binding efficiency of oligonucleotides comprising one or more modified oligonucleotides, modified oligonucleotides were generated and compared to the hybridization efficiency of an unmodified oligonucleotide. The binding efficiency was determined relative to the pH of the hybridization reaction.

The oligonucleotides contain one or both of the modified bases 5-hydroxycytosine (C*) and 8-hydroxyguanine (G*). An unmodified oligonucleotide was used as a control. The oligonucleotides are set out in Table 1.

TABLE 1

| OLIGONUCLEOTIDE | SEQUENCE |
|---|---|
| f | TCAGAACTTCAAAACTACTTC |
| | (SEQ ID NO 1) |
| f1 | TCAGAACTTCAAAACTA(C*)TTC |
| | (SEQ ID NO 2) |
| f2 | TCAGAACTTCAAAA(C*)TACTTC |
| | (SEQ ID NO 3) |
| f3 | TCA(G*)AACTTCAAAACTACTTC |
| | (SEQ ID NO 4) |
| f* | TCAGAACTTCAAA(A*)CTA(C*)TTC |
| | (SEQ ID NO 5) |

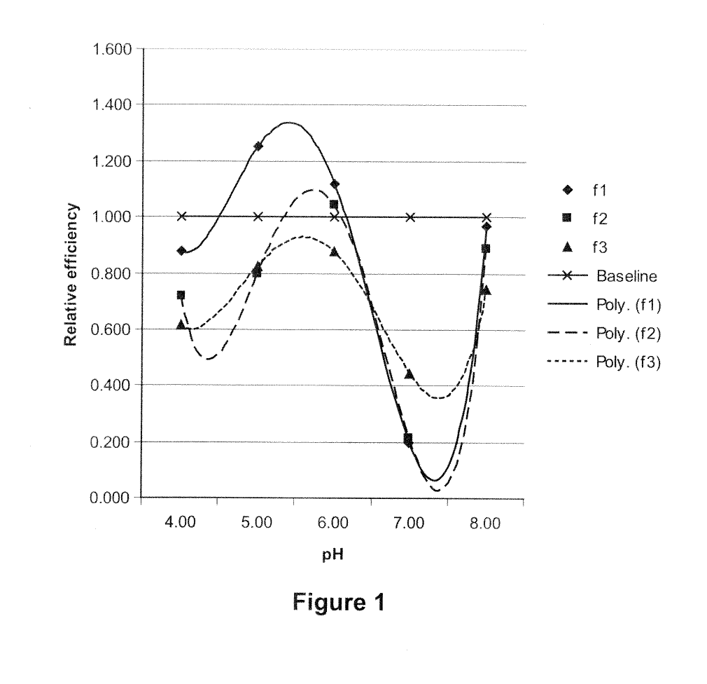

The pH-dependence of the relative binding efficiency of the modified oligonucleotides was studied at different target amounts/concentrations. The data on the modified oligonucleotide binding efficiency as related to the binding efficiency of native oligonucleotide at the concentration of complementary oligonucleotide on the membrane of 1 pmol were fitted using the 4^{th} order polynomial and the respective graphs are given in FIG. 1. The relative binding is efficiency defined as

$$E = \frac{{D}_{mod}}{{D}_{nat}}$$

where D_{mod} is the binding efficiency of the oligonucleotide with modified nucleobases and Dₙₐₜ denotes the binding efficiency of the native oligonucleotide.

The data presented in FIG. 1 demonstrate a strong dependence of the binding efficiency of the modified oligonucleotides on pH. This is due to the presence of tautomeric equilibria, as the anion of the modified base 5-hydroxycytosine and 8-hydroxyguanine exist in several major prototropic tautomeric forms (Schemes 1 and 2), the ratio of which can substantially depend on the pH of the surrounding medium (solution). The binding efficiency of different tautomeric forms can be remarkably different, leading to a strong dependence of the observable binding efficiency on pH.

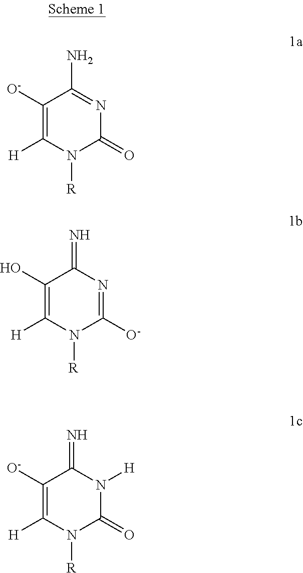

In the case of 5-hydroxycytosine anion, the tautomeric form 1b has the binding efficiency to complementary guanine base of about 10⁷ times higher than cytosine itself. The tautomeric form 1a is expected to have a similar binding efficiency to cytosine whereas the tautomeric form 1c has a diminished binding with the complementary base.

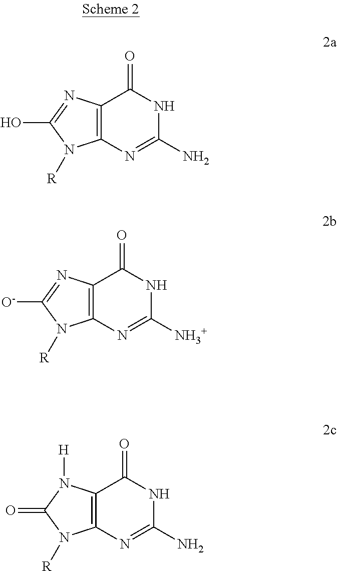

In the case of 8-hydroxyguanine, the tautomeric form 2b has again much higher binding efficiency to complementary cytosine base (about 10⁷-10⁸ times higher) than guanine itself. The tautomeric forms 2a and 2c are expected to have a smaller binding efficiency as compared to guanine.

The pH-dependence of the relative binding efficiency of modified oligonucleotides has a well-defined maximum at pH values around 5.0-6.0, followed with a sharp minimum at about pH=7.5. The further increase of pH leads again to a substantial increase of the relative binding efficiency of modified oligonucleotides. The maximum efficiency depends both on the nature of the modification (5-hydroxycytosine or 8-hydroxyguanine) and the position of the substitution in the oligomer (compare oligonucleotides f1 and f2). The highest efficiency was observed for modified oligonucleotide f1 (1.37).

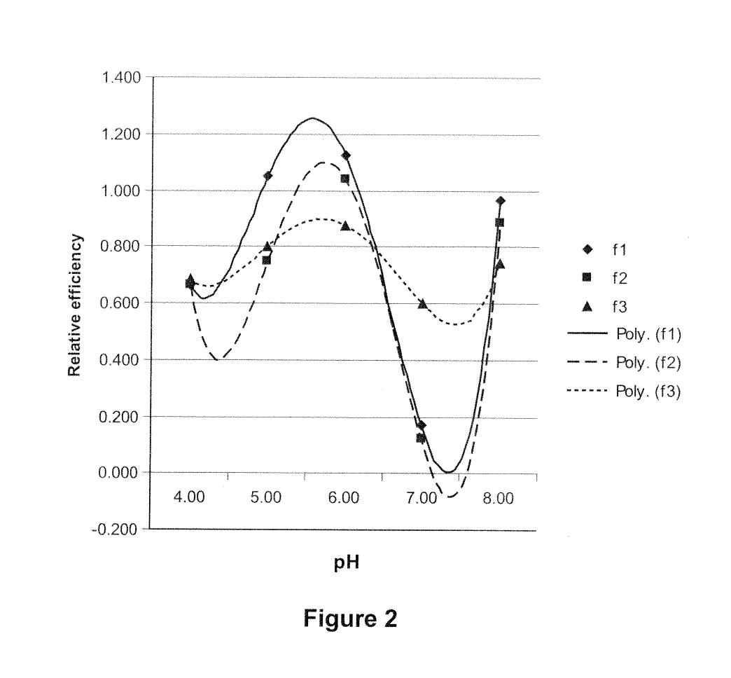

Analogous pH-dependencies of the relative binding efficiency of modified oligonucleotides were obtained at the oligonucleotide concentrations 5 pmol (FIG. 2) and 25 pmol (data not shown).

### Example 2

### Binding of Modified/Unmodified Antisense Oligonucleotides to the Complementary Membrane-Bound DNA

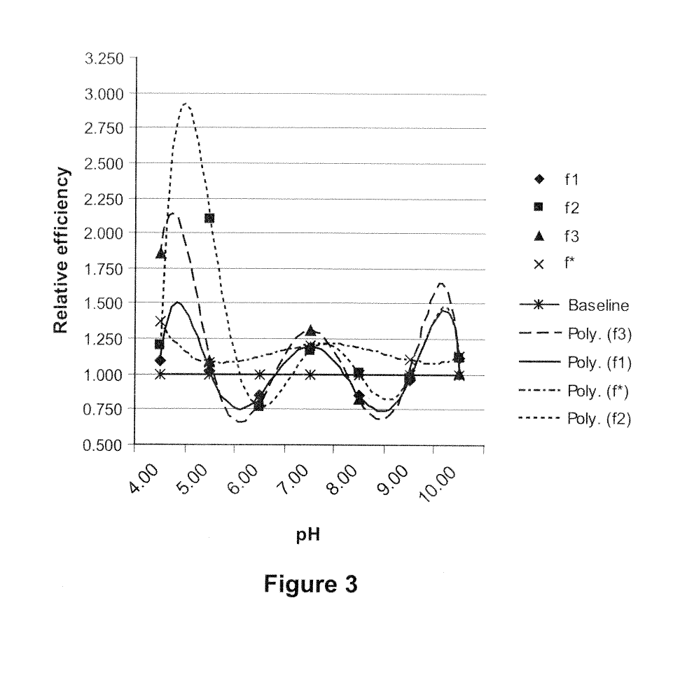

Similar experiments to measure the relative binding efficiency of oligonucleotides with DNA were carried out using the same methodology. The data for each modified oligonucleotide were again fitted using the 4^{th} order polynomial and the respective graphs are given in FIG. 3.

In contrast to the oligonucleotide-oligonucleotide binding, in this case the pH dependence has three maxima, the highest of which is, however, again at lower pH values (around pH=4.5 . . . 5.0). This maximum is also significantly higher than for the oligonucleotide-oligonucleotide binding. For modified oligonucleotides f2 and f1, the relative binding efficiencies are 2.9 and 2.1, respectively. The other two maxima in the pH-dependence of the relative binding efficiency of modified oligonucleotides are localized at around pH=7.5 and pH=10, respectively (FIG. 3).

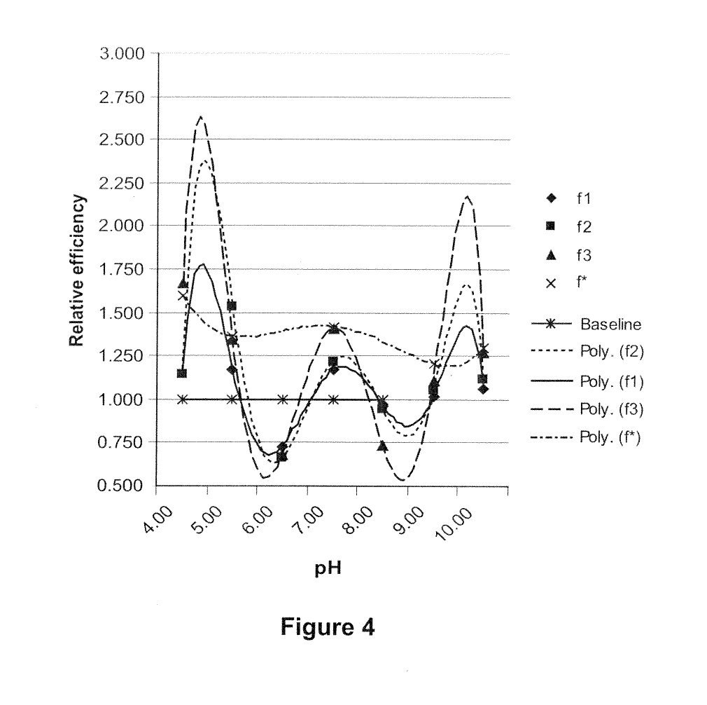

Remarkably, the oligonucleotide f* with two modifications (both 5-hydroxycytosine and 8-hydroxyguanine involvement) has much smoother dependence of the relative binding efficiency on pH (FIGS. 3 and 4).

A similar pH-dependence of the relative binding efficiency was observed at the amount of DNA 5 ng on the membrane (FIG. 4).

### Example 3

### Usage of Modified Oligonucleotides in Hybridization

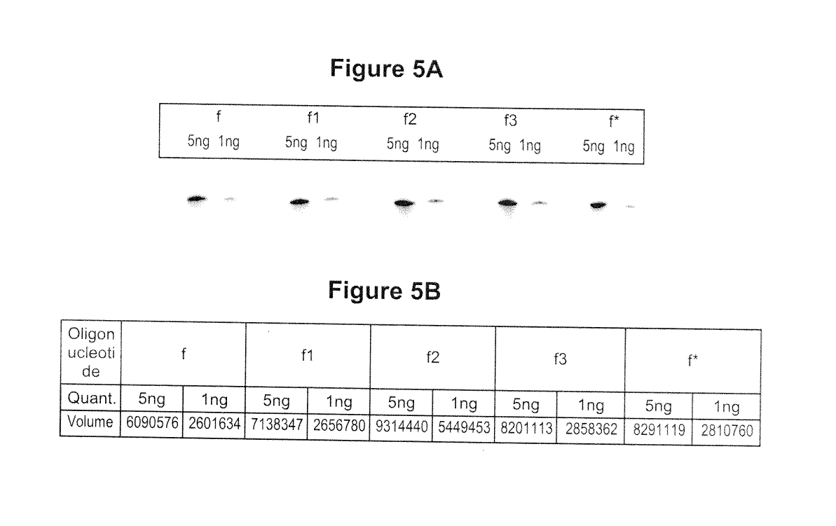

On FIG. 5 panel A, 5 ng and 1 ng of a 2 kb long cDNA of Arabidopsis thaliana RLI2 gene was analyzed by electrophoresis under denaturating conditions on a 5% polyacrylamide gel in TBE buffer. The gel was electroblotted onto a nylon membrane and the DNA immobilized by UV crosslinking The membranes were hybridized over night at 45° C. in 6×SSC, 2× Denhardt's solution, 0.1% SDS and pH 5.0 with five ³²P 5′ labeled oligonucleotides which were all identical in sequence. The probes were: f, f1, f2, f3 and f* (cf. Table 1). The membranes were washed for two times for 10 minutes at 45° C. with 2×SSC, 0.5% SDS pH 5.0. Radioactive signal was detected with Molecular Imager Personal FX (BioRad). Panel B, the detected signals shown in A were quantified with ImageQuant TL (Amersham) software. The respective data are given on FIG. 5.

### Example 4

### Binding of Modified/Unmodified Antisense Oligonucleotides to Complementary Membrane-Bound mRNA

The experiments to measure the relative binding efficiency of oligonucleotides containing the modified nucleobases (5-hydroxycytosine and 5-hydroxyguanine) with the complementary native mRNA immobilized on the membrane were carried out using the same methodology. The data on the pH-dependence of the relative binding efficiency of the mRNA with modified nucleotides has two distinct maxima, the highest of which is localized around pH=5.8 . . . 6.2 (FIG. 5).

In this case, the highest efficiency at the maximum is exhibited by the oligonucleotide containing modified guanine nucleobases (8-hydroxyguanine)—1.5 times higher efficiency than the non-modified oligonucleotide.

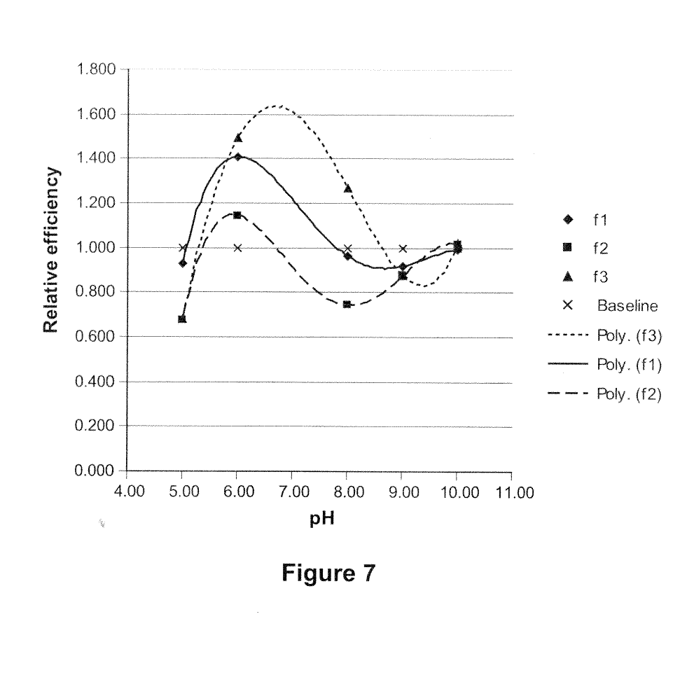

An analogous pH-dependence of the relative binding efficiency of the modified oligonucleotides was observed for the amount of mRNA on the membrane 2.5 ng, with the maxima shifted somewhat to more alkaline side (FIG. 7).

### Example 5

### The Dependence of the Relative Binding Efficiency of the Modified Oligonucleotides on the Target Concentration

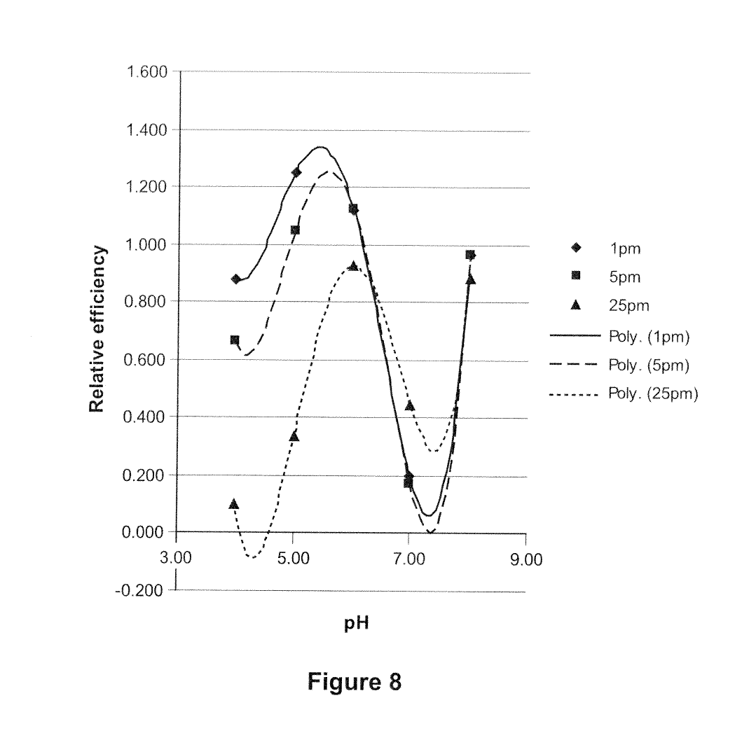

The existence of tautomeric forms with higher binding efficiency in oligonucleotides can lead to their higher relative binding efficiencies at lower concentrations. In the oligonucleotide-oligonucleotide experiments, when the sense and antisense concentrations are similar, such behavior is observed. In FIG. 8, the pH-dependence of the relative binding efficiency of modified oligonucleotide fl is presented at different target concentrations (1 pmol, 5 pmol, 25 pmol). Consequently, the relative binding efficiency increases with the lowering of the target concentration.

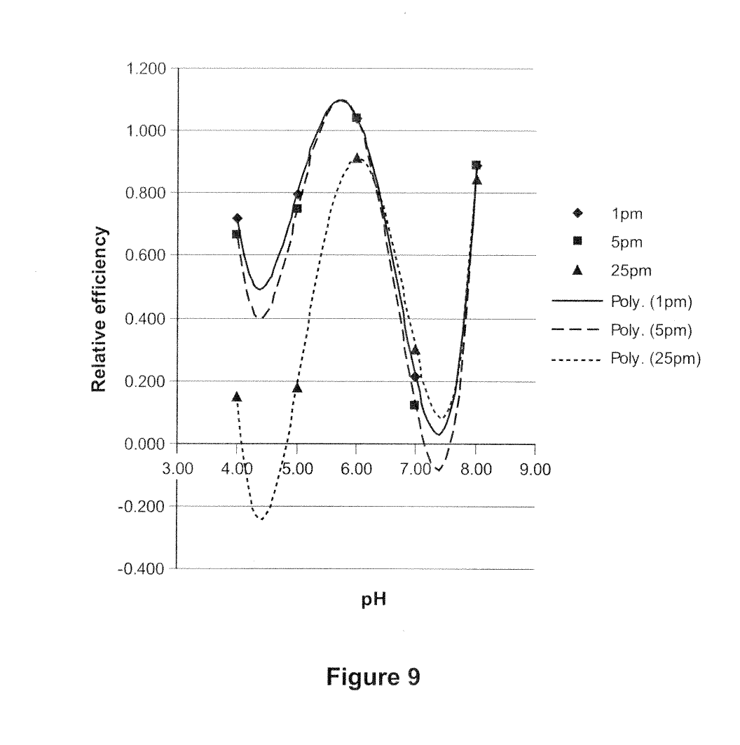

A similar dependence is observed in the case of modified oligonucleotide f2 (FIG. 9).

Conclusions from Hybridization Experiments.

The binding of oligonucleotides containing tautomeric modified nucleobases (5-hydroxycytosine and/or 8-hydroxyguanine) to complementary sequences in oligonucleotides, DNA and mRNA as compared to native oligonucleotides has complex dependence on pH. Typically, there are 2 or 3 distinct maxima in the relative binding efficiency D. The highest relative efficiency is localized at acidic pH value around pH=4.8 . . . 6.2, depending on the system used.

The relative binding efficiency depends both on the nature of the modification as well as on the nature of the counterpart (oligonucleotide, DNA or RNA). The presently found highest relative efficiencies are between 1.5 . . . 3 as compared to the binding efficiencies of the unmodified oligonucleotide.

### Example 6

### Modified Oligonucleotides as PCR Primers at Different Annealing Temperatures

A 383 by long fragment of Arabidopsis thaliana RLI2 DNA sequence was amplified by PCR reaction at annealing temperatures 47, 48.7, 51.3, 58.4, 61.7, 64.3, 66.1, 67.5 and 68° C. The fragment was amplified with unmodified oligonucleotides F (5′-TCAGAACTTCAAAACTACTTC (SEQ ID NO 1), corresponding to nt 1638-1658 in AtRLI2 coding sequence) and R (5′-TTCATCAAACATGTAAATCTC (SEQ ID NO 6), corresponding to nt 2001-2021 in AtRLI2 coding sequence in reverse complement orientation); and in parallel with oligonucleotides containing two modifications identical in sequence to F and R marked as f* (5′-TCAGAACTTCAAAACTACTTC (SEQ ID NO 5), modified bases are underlined) and r* (5′-TTCATCAAACATGTAAATCTC (SEQ ID NO 7), modified bases are underlined). PCR mixture contained 20 pmol of each primer, the final Mg²⁻ concentration was 2.5 mM and 25 ng of the same 383 bp long fragment was used as a template. PCR program consisted of the initial denaturation step (2 min at 95° C.), followed by 30 cycles of denaturation (40 sec at 95° C.), annealing (40 sec at 47-68° C.) and polymerization (40 sec at 72° C.). The final step of PCR was 10 minutes at 72° C. The products were separated by electrophoresis in a 1.7% agarose gel in TAE buffer and visualized by ethidium bromide staining and UV light.

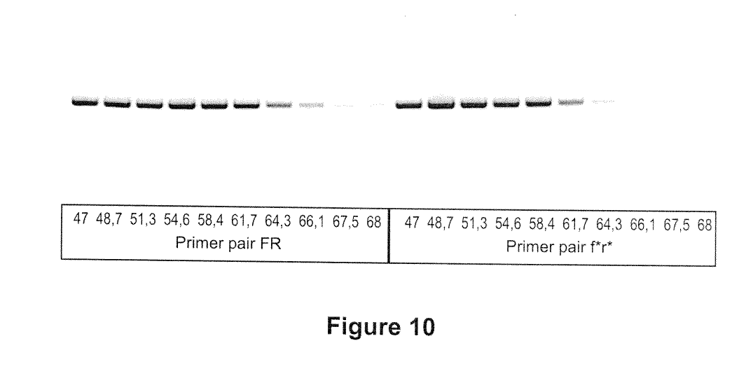

The results given in FIG. 10 demonstrate the applicability and efficacy of the oligonucleotide with modified bases in PCR.

### Example 7

### Modified Oligonucleotides as siRNAs in Transfected Cells

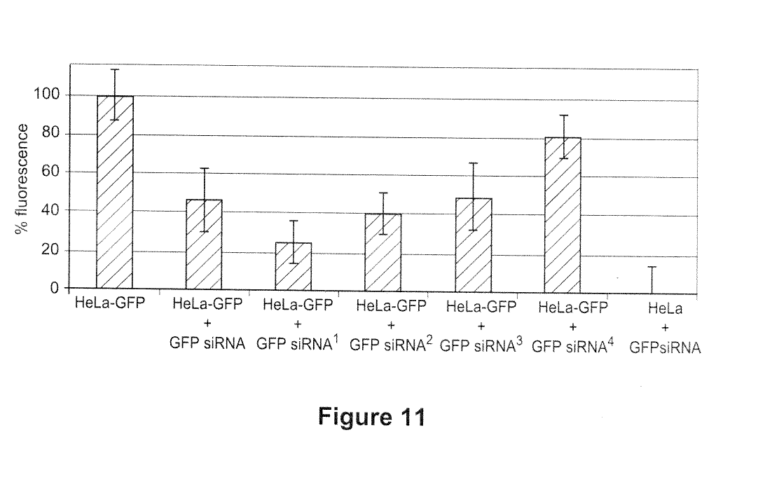

Effect of modified siRNAs on eGFP transgene expression was measured as follows. HeLa-GFP (green fluorescent protein) stable transgenic line was grown on DMEM with 5% serum and 4 mg/ml G418 (Geneticin, Sigma). Transfection with GFP siRNAs was carried out as described in Lipofectamine 2000 (Invitrogen) protocol in a 24 well-plate (75,000 cells per well) using DMEM instead of Opti-MEM. Three days after transfection, cells were resuspended in PBS and fluorescence levels (emission at 535 nm) were analyzed by GENios Pro TECAN. The eGFP expression is shown as the percentage of the fluorescence level of the HeLa-GFP mock-transfected line (with negative control siRNA Alexa Fluor 546, Qiagen) (FIG. 11). Non-transgenic HeLa cells transfected with GFP siRNA were used as negative control. Percentages and SD are calculated from 4 parallel transfections. Underlined nucleotides were modified.

| GFP | commercial “GFP-22 siRNA” | SEQ |
|---|---|---|
| siRNA: | (Qiagen) | ID NO |
| | 5′ GCAAGCUGACCCUGAAGUUCAU 3′ | 8 |
| | 3′GCCGUUCGACUGGGACUUCAAG 5′ | 9 |
| GFP | 5′ GCAAGCUGACCCUGAAGUUCAU 3′ | 8 |
| siRNA¹: | 3′GCCGUUCGACUGGGACUUCAAG 5′ | 10 |
| GFP | 5′ GCAAGCUGACCCUGAAGUUCAU 3′ | 8 |
| siRNA²: | 3′GCCGUUCGACUGGGACUUCAAG 5′ | 11 |
| GFP | 5′ GCAAGCUGACCCUGAAGUUCAU 3′ | 8 |
| siRNA³: | 3′GCCGUUCGACUGGGACUUCAAG 5′ | 12 |
| GFP | 5′ GCAAGCUGACCCUGAAGUUCAU 3′ | 8 |
| siRNA⁴: | 3′GCCGUUCGACUGGGACUUCAAG 5′ | 13 |

### Example 8

### Effect of Modifications on Hybridization Kinetics

Possible differences in hybridization kinetics between modified and unmodified oligonucleotides were studied as follows. 0.5, 1, 2.5, 5 and 10 ng of target DNA (in 2×SSC) was dot blotted onto nylon membrane and hybridized over night at 45° C. in 6×SSC, 2× Denhardt's solution, 0.1% SDS and pH 5.0 with five ³²P 5′ labeled oligonucleotides which were all identical in sequence. The probes were: f, f1, f2, f3 and f8 (cf. Table 1). Time points were taken with the interval of 10 minutes, from the 10^{th} minute up to the 60^{th} minute, after the addition of the probe. The membranes were washed for two times for 10 minutes at room temperature with 2×SSC, 0.5% SDS pH 5.0. Radioactive signal was detected with Molecular Imager Personal FX (BioRad). Pseudo-first order hybridization rate constants were calculated using 1nV-t method. The results (smoothed relative rate constants) are given in Table 2. All modified oligonucleotides have higher hybridization rates than unmodified oligonucleotides, especially at smaller substrate quantities.

TABLE 2
Relative hybridization rate constants kᵣₑₗ of modified
oligonucleotides against unmodified oligonucleotides.

| | Substrate amount mₛ (ng) | | | | |
|---|---|---|---|---|---|
| Oligonucleotide | 0.5 | 1.0 | 2.5 | 5.0 | 10.0 |
| f | 1.00 | 1.00 | 1.00 | 1.00 | 1.00 |
| f1 | 2.33 | 2.02 | 1.75 | 1.28 | 1.26 |
| f2 | 1.70 | 1.73 | 1.47 | 1.07 | 1.15 |
| f3 | 3.40 | 2.55 | 2.43 | 1.97 | 1.93 |
| f* | 1.88 | 2.25 | 2.33 | 1.87 | 1.73 |

1. A method of inhibiting expression of a target nucleic acid comprising:
contacting a target nucleic acid of known sequence with a modified oligonucleotide having a sequence of nucleobases that is at least partly complementary to a strand of said target nucleic acid, under conditions that permit hybridizing of the modified oligonucleotide to a strand of thc target nucleic acid,
wherein the hybridized modified oligonucleotide inhibits expression of the target nucleic acid,
wherein the modified oligonucleotide comprises from 5 to 150 nucleobases, and
wherein at least one of the nucleobases of the modified oligonucleotide is a modified nucleobase selected from the group consisting of: 5-mercaptocytosine, 5-mercaptouracil, 8-mercaptoguanine, 8-mercaptoadenine, 5-hydroxycytosine, 5-hydroxyuracil, 8-hydroxyadenine and 8-hydroxyguanine.

2. The method of claim 1 wherein expression is inhibited by at least 20%.

3. The method of claim 1, wherein the modified oligonucleotide is RNA.

4. The method of claim 3 wherein the RNA is single-stranded.

5. The method of claim 3 wherein the RNA is double-stranded, and wherein at east one strand of the RNA comprises the at least one modified nucleobase.

6. The method of claim 1, wherein the target nucleic acid is in a cell, and the contacting comprises introducing the modified oligonucleotide into the cell.

7. The method of claim 6 wherein the contacting is selected from the group consisting of transforming and transfecting the cell with the modified oligonucleotide.

8. The method of claim 1, wherein the target nucleic acid is in a cell of an organism, and wherein the contacting comprises administering to the organism a composition that comprises the modified oligonucleotide and a pharmaceutically acceptable carrier.

9. The method of claim 8 wherein the organism is mammal.

10. The method of claim 9 wherein the organism is a human.

11. A method of detecting a target nucleic acid with a modified oligonucleotide comprising:
contacting a target nucleic acid with a modified oligonucleotide under conditions that permit hybridizing of the modified oligonucleotide to a strand of said target nucleic acid,
wherein the modified oligonucleotide comprises a sequence of nucleobases that is at least partly complementary to a sequence of the strand of the target nucleic acid, and
wherein at least one of the nucleobases of the modified oligonucleotide is a modified nucleobase selected from the group consisting of: 5-mercaptocytosine, 5-mercaptouracil, 8-mercaptoguanine, 8-mercaptoadenine, 5-hydroxycytosine, 5-hydroxyuracil, 8-hydroxyadenine and 8-hydroxyguanine; and
detecting the target nucleic acid by detecting the modified oligonucleotide hybridized to the strand of the target nucleic acid.

12. The method of claim 11, wherein the target nucleic acid is immobilized to a solid support.

13. The method of claim 12 wherein the immobilized target nucleic acid is DNA.

14. The method of claim 12 wherein the immobilized target nucleic acid is RNA.

15. The method of claim 11, wherein the detecting is quantitative.

16. A method of polymerase chain reaction (PCR) comprising:
contacting a template nucleic acid with a modified oligonucleotide comprising a sequence sufficiently complementary to a portion of the template nucleic acid to allow hybridization of the modified oligonucleotide with the template nucleic acid under PCR annealing conditions,
wherein the hybridized modified oligonucleotide serves as a PCR primer under PCR amplification conditions to generate a first strand PCR product, and
wherein the modified oligonucleotide comprises from 5 to 150 nucleobases, wherein at least one of the nucleobases is a modified nucleobase selected from the group consisting of: 5-mercaptocytosine, 5-mercaptouracil, 8-mercaptoguanine, 8-mercaptoadenine, 5-hydroxycylosine, 5-hydroxyuracil, 8-hydroxyadenine and 8-hydroxyguanine and;
performing a polymerasc chain reaction (PCR) with the modified oligonucleotide and the template nucleic acid.

17. The method of claim 16, wherein the PCR comprises:
making a reaction mixture that contains a thennostable DNA polymerase, the template nucleic acid, the modified oligonucleotide, and nucleotides.

18. The method of claim 17, wherein the PCR reaction mixture further comprises a second oligonucleotide that comprises a nucleotide sequence complementary to a part of a strand of the target nucleic acid or a part of the first strand PCR product.

19. The method of claim 18, wherein the second oligonucleotide is a modified oligonucleotide, wherein the modified oligonucleotide comprises from 5 to 150 nucleobases, wherein at least one of the nucleobases is a modified nucleobase selected from the group consisting of: 5-mercaptocytosine, 5-mercaptouracil, 8-mercaptoguanine, 8-mercaptoadenine, 5-hydroxycytosine, 5-hydroxyuracil, 8-hydroxyadenine and 8-hydroxyguanine.

20. The method of claim 16 wherein the template nucleic acid is DNA.

21. The method of claim 16 wherein the template nucleic acid is RNA.

22. The method of claim 16 wherein the amplified product is quantified in real time.

23. The method of claim 16, wherein the polymerase chain reaction comprises repeated steps of denaturing the template nucleic acid, annealing the modified oligonucleotide and template nucleic acid under annealing conditions, and synthesizing a polymerase chain reaction product by extending the annealed modified oligonucleotide.

24. The method of claim 11 wherein the modified oligonucleotide comprises a detectable label.

25. The method of claim 1 wherein the hybridizing conditions comprise a pH of between 4 and 10.

26. The method of claim 25 wherein the is between 4 and 6.

27. The method of any one of claim 1 wherein the modified oligonucleotide has a length of from 10 to 100 nucleobases.

28. The method of claim 1 wherein the modified oligonucleotide has a length of from 10 to 50 nucleobases.

29. The method of claim 1 wherein the modified oligonucleotide has a length of from 20 to 30 nucleobases.

30. The method of claim 1 wherein from 0.5% to 40% of the nucleobases of the modified oligonucleotide comprise mercapto- or hydroxynucleobases.

31. In a method of amplification of a target nucleic acid, the method comprising annealing an oligonucleotide primer that is complementary to at least a portion of the target nucleic acid to the target nucleic acid, the improvement comprising using a modified oligonucleotide as the oligonucleotide primer, wherein the modified oligonucleotide comprises one or more modified nucleobases selected from the group consisting of: 5-mercaptocytosine, 5-mercaptouracil, 8-mercaptoguanine, 8-mercaptoadenine, 5-hydroxycytosine, 5-hydroxyuracil, 8-hydroxyadenine and 8-hydroxyguanine.

32. The improvement of claim 31, wherein the method of amplification is an exponential amplification method.

33. The improvement of claim 31, wherein the method of amplification is polymerase chain reaction (PCR).

34. The method of claim 11, comprising selecting a pH at which the modifiedoligonucleotide exhibits improved relative binding to the target nucleic acid, compared to an unmodified oligonucleotide having an identical sequence, and performing the contacting at the selected pH.

35. An isolated nucleic acid consisting of a polynucleotide sequence selected from the group consisting of SEQ ID NO: 10, SEQ ID NO: 11, SEQ ID NO: 12, and SEQ ID NO: 13.


SEQUENCE LISTING (13 sequences)

SEQ ID NO: 1 - DNA, 21 bp, Artificial Sequence
  Synthetic Oligonucleotide
tcagaacttcaaaactacttc

SEQ ID NO: 2 - DNA, 21 bp, Artificial Sequence
  Synthetic Oligonucleotide
tcagaacttcaaaactacttc

SEQ ID NO: 3 - DNA, 21 bp, Artificial Sequence
  Synthetic Oligonucleotide
tcagaacttcaaaactacttc

SEQ ID NO: 4 - DNA, 21 bp, Artificial Sequence
  Synthetic Oligonucleotide
tcagaacttcaaaactacttc

SEQ ID NO: 5 - DNA, 21 bp, Artificial Sequence
  Synthetic Oligonucleotide
tcagaacttcaaaactacttc

SEQ ID NO: 6 - DNA, 21 bp, Artificial Sequence
  Synthetic Oligonucleotide
ttcatcaaacatgtaaatctc

SEQ ID NO: 7 - DNA, 21 bp, Artificial Sequence
  Synthetic Oligonucleotide
ttcatcaaacatgtaaatctc

SEQ ID NO: 8 - RNA, 24 nt, Artificial Sequence
  Synthetic RNA
cggcaagcugacccugaaguucau

SEQ ID NO: 9 - RNA, 22 nt, Artificial Sequence
  Synthetic RNA
gaacuucagggucagcuugccg

SEQ ID NO: 10 - RNA, 22 nt, Artificial Sequence
  Synthetic RNA
gaacuucagggucagcuugccg

SEQ ID NO: 11 - RNA, 22 nt, Artificial Sequence
  Synthetic RNA
gaacuucagggucagcuugccg

SEQ ID NO: 12 - RNA, 22 nt, Artificial Sequence
  Synthetic RNA
gaacuucagggucagcuugccg

SEQ ID NO: 13 - RNA, 22 nt, Artificial Sequence
  Synthetic RNA
gaacuucagggucagcuugccg